(19) United States
(12) Patent Application Publication
(10) Pub. No.: US 2015/0313502 A1
(43) Pub. Date: Nov. 5, 2015

(54) DETERMINING ARTERIAL PULSE WAVE TRANSIT TIME FROM VPG AND ECG/EKG SIGNALS

(71) Applicant: XEROX CORPORATION, Norwalk, CT (US)

(72) Inventors: Lalit Keshav MESTHA, Fairport, NY (US); Survi KYAL, Rochester, NY (US)

(73) Assignee: XEROX CORPORATION, Norwalk, CT (US)

(21) Appl. No.: 14/268,656

(22) Filed: May 2, 2014

(51) Int. Cl. A61B 5/0452; A61B 5/117; A61B 5/00; A61B 5/0265; A61B 5/021; A61B 5/0295; A61B 5/0205; A61B 5/026; A61B 5/02

(52) U.S. Cl. CPC A61B 5/04525; A61B 5/0261; A61B 5/1176; A61B 5/6898; A61B 5/7207; A61B 5/02007; A61B 5/02125; A61B 5/0295; A61B 5/0006; A61B 5/0013; A61B 5/0022; A61B 5/0205; A61B 5/0265; A61B 2576/00; A61B 2560/0475; A61B 5/02433

(57) ABSTRACT

What is disclosed is a system and method for determining arterial pulse wave transit time for a subject. In one embodiment, a video is received comprising a plurality of time-sequential image frames of a region of exposed skin of a subject where a videoplethysmographic (VPG) signal can be registered by at least one imaging channel of the video device used to capture that video. Also received is an electrocardiogram (ECG) signal obtained using at least one sensor placed on the subject's body where a ECG signal can be obtained. Batches of image frames are processed to obtain a continuous VPG signal for the subject. Temporally overlapping VPG and ECG signals are analyzed to obtain a pulse wave transit time between a reference point on the VPG signal and a reference point on the ECG signal. The pulse transit time is used to assess pathologic conditions such as peripheral vascular disease.

### TECHNICAL FIELD

The present invention is directed to systems and methods for determining an arterial pulse wave transit time for a subject.

### BACKGROUND

The ability to capture physiological signals is highly desirable in the healthcare industry. One physiological signal of importance is the arterial pulse transit time (PTT). This is important for many reasons, one of which is that the PTT correlates well with blood pressure and thus can provide healthcare professionals with vital information relating to blood velocity in the vascular network, blood vessel dilation over time, and vessel blockage between two points or regions. Moreover, localized PTT can be used as an indirect marker for assessing pathologic conditions such as peripheral vascular disease.

Accordingly, what is needed in this art are increasingly sophisticated systems and methods for determining a subject's arterial pulse transit time.

### INCORPORATED REFERENCES

The following U.S. patents, U.S. patent applications, and Publications are incorporated herein in their entirety by reference.
“Deriving Arterial Pulse Transit Time From A Source Video Image”, U.S. patent application Ser. No. 13/401,286, by Mestha.
“Determining Cardiac Arrhythmia From A Video Of A Subject Being Monitored For Cardiac Function”, U.S. patent application Ser. No. 14/245,405 by Mestha et al.
“System And Method For Determining Video-Based Pulse Transit Time With Time-Series Signals”, U.S. patent application Ser. No. 14/026,739, by Mestha et al.
“System And Method For Determining Arterial Pulse Wave Transit Time”, U.S. patent application Ser. No. 14/204,397, by Mestha et al.
“Cardiac Pulse Rate Estimation From Source Video Data”, U.S. patent application Ser. No. 14/200,759, by Kyal et al.

“Determining A Total Number Of People In An IR Image Obtained Via An IR Imaging System”, U.S. Pat. No. 8,520,074, by Wang et al.
“Determining A Number Of Objects In An IR Image”, U.S. Pat. No. 8,587,657, by Wang et al.
“Determining A Pixel Classification Threshold For Vehicle Occupancy Detection”, U.S. patent application Ser. No. 13/324,308, by Wang et al.

### BRIEF SUMMARY

What is disclosed is a system and method for determining arterial pulse transit time. In one embodiment, a video is received comprising a plurality of time-sequential image frames of a region of exposed skin of a subject where a videoplethysmographic (VPG) signal can be registered by at least one imaging channel of the video device used to capture that video. Also received is an electrocardiogram (ECG) signal obtained using a sensor placed on the subject's body where a ECG signal can be obtained. Batches of image frames are processed to obtain a continuous VPG signal for the subject. Thereafter, temporally overlapping VPG and ECG signals are analyzed to obtain the pulse transit time between a reference point on the VPG signal and a reference point on the ECG signal. The pulse transit time can be used to assess pathologic conditions such as peripheral vascular disease.

Features and advantages of the above-described method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 shows a video image device capturing real-time video of a subject;

FIG. 2 shows a batch of image frames of the video acquired by the video imaging device of FIG. 1;

FIG. 3 shows one of the image frames of the batch of FIG. 2 with various regions of exposed skin having been identified for processing;

FIG. 4 shows a portion of an ECG signal of a normal sinus rhythm obtained by the electrocardiogram of FIG. 1;

FIG. 5 is a flow diagram which illustrates one example embodiment of the present method for determining arterial pulse transit time for a subject;

FIG. 6 illustrates a block diagram of one example signal processing system 600 for performing various aspects of the teachings hereof;

FIG. 7 shows a 10 second portion of a VPG signal overlaid on a simultaneously acquired ECG signal; and

FIG. 8 shows the normalized power spectral density for ECG and VPG signals v/s cardiac pulse frequency in beats per minute.

### DETAILED DESCRIPTION

What is disclosed is a system and method for determining arterial pulse wave transit time for a subject.

### Non-Limiting Definitions

“Plethysmography” is the study of relative blood volume changes in blood vessels.

A “photoplethysmographic (PPG) signal” is a plethysmographic signal obtained using optical instruments.

A “videoplethysmographic (VPG) signal” is a plethysmographic signal extracted from video.

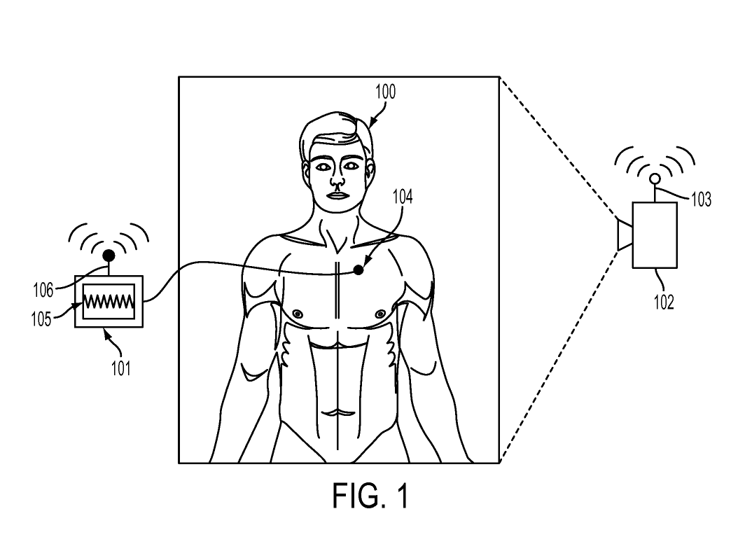

A “subject” is a living being. One example subject 100 is shown in FIG. 1. Although the term “person” or “patient” may be used throughout this disclosure, it should be appreciated that the subject may be something other than a human such as, for example, a primate. Therefore, the use of such terms is not to be viewed as limiting the scope of the appended claims strictly to humans.

A “video”, as is generally understood, is a time-varying sequence of image frames acquired by a video imaging device. The video may contain other components such as, audio, time reference signals, frame rate, and the like.

A “video imaging device” is a single-channel or a multi-channel device for capturing video. FIG. 1 shows an example video imaging device 102 actively acquiring video 101 of a subject 100. Image frames of the video may be communicated to a remote device via a wireless communication element 103, shown as an antenna. In one embodiment, the video imaging device has a high frame rate and high spatial resolution such as, for example, a monochrome camera for capturing black/white video, or a color camera for capturing color video. In another embodiment, the video imaging device is a device with thermal, infrared, multi-spectral or hyperspectral sensors. In yet another embodiment, the video imaging device is a hybrid device capable of operating in a conventional video mode with high frame rates and high spatial resolution, and a spectral mode with low frame rates but high spectral resolution. The video imaging device typically has a plurality of outputs for retrieving the image frames on a per-channel basis and may further incorporate other components such as memory, one or more storage devices. Video imaging devices may incorporate one or more processors executing machine readable program instructions for analyzing batches of image frames in real-time, in accordance with the teachings hereof. Video imaging devices comprising standard video equipment and those with specialized imaging sensors are readily available from vendors in various streams of commerce.

“Receiving image frames” is intended to be widely construed and includes: retrieving, capturing, acquiring, or otherwise obtaining image frames for processing to obtain a VPG signal for the subject. The image frames can be retrieved from a memory or storage device of the video imaging device or retrieved from a media such as a CDROM or DVD. Image frames can be obtained from a remote device over a network or downloaded from a web-based system or application which makes image frames available for processing. The image frames may be processed to compensate for motion induced blur, imaging blur, and slow illuminant variation. The video is preferably processed in overlapping batches of time-sequential image frames.

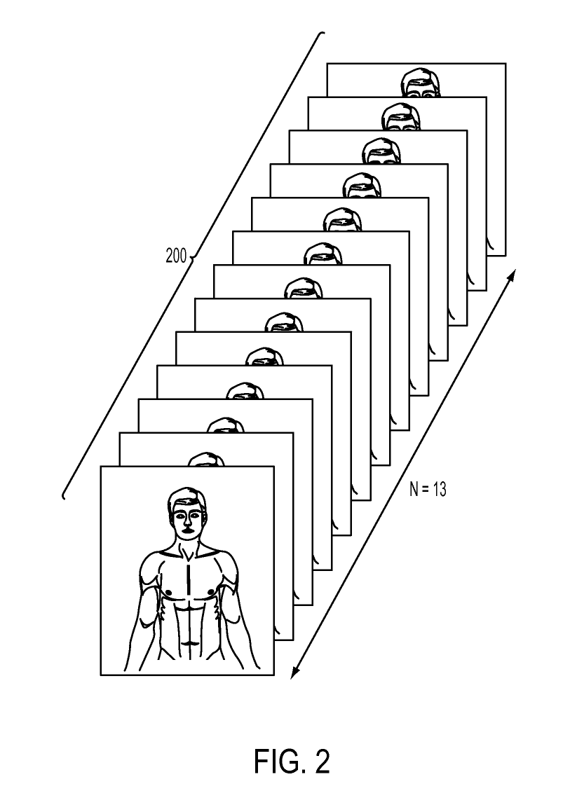

A “batch of image frames” refers to a plurality of time-sequential image frames. FIG. 2 shows an example batch of 13 image frames (collectively at 200) acquired by the video imaging device 102 of FIG. 1. Batches of image frames do not have to be the same size and may vary dynamically during processing. A size of a given batch of video image frames should at least be of a duration which captures one cardiac cycle of the subject. Batches of image frames can be defined for processing utilizing a sliding window. In one example, the sliding window defines 1 second of new image frames overlapping 29 seconds of image frames from the previous batch, (i.e., a 96% overlap). The size of the sliding window which may be dynamically adjusted in real-time as needed. Image frames of a given batch are processed to isolate a region of exposed skin.

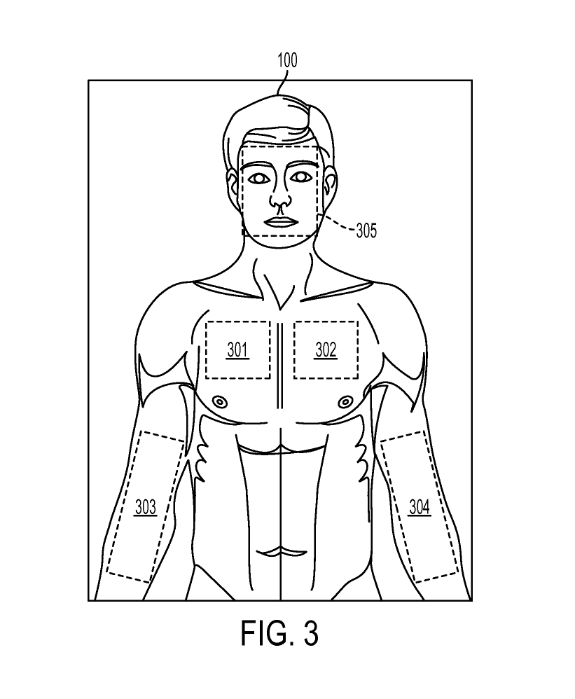

“A region of exposed skin” refers to an unobstructed view of the subject's skin as seen through the lens of the video imaging device. FIG. 3 shows one of the image frames of the batch of FIG. 2 with a box around various regions of exposed skin (301, 302, 303, 304 and 305). It should be appreciated that the regions of exposed skin of FIG. 3 are for explanatory purposes and that other regions of exposed skin may be identified or otherwise selected. FIG. 3 should not be viewed as limiting the scope of the appended claims solely to the illustrated regions. A region of exposed skin can be identified in a given image frame using a wide array of image processing techniques which include, for example, color and texture identification, object identification, thoracic region recognition, spatial feature analysis, spectral information, pattern recognition, face detection methods, and facial recognition algorithms. Moreover, a user or technician may use a mouse or, for instance, a touchscreen display to identify one or more regions of exposed skin in the image frames of the video for pixel isolation. Regions of exposed skin do not have to be the same size. The size of a given region of exposed skin will vary depending on the application. The lens of the video camera is preferably zoomed-in on the subject to capture a large enough region of exposed skin to obtain a sufficient numbers of pixels of skin surface for processing. Pixels in a region of exposed skin are isolated for processing.

“Isolating pixels” in a region of exposed skin can be effectuated using techniques which include pixel classification, object identification, thoracic region recognition, color, texture, spatial features, spectral information, pattern recognition, face detection, facial recognition, and a user input. Methods for classifying pixels in an image are disclosed in several of the above-incorporated references by Wang et al. Pixels may be weighted, averaged, normalized, or discarded, as needed. Isolated pixels are processed to obtain a time-series signal.

A “time-series signal” is a signal that contains frequency components that relate to the subject's cardiac function. More specifically, the time-series signal contains the sum total of the relative blood volume changes in the blood vessels close to the skin surface within the isolated region of exposed skin. These arterial pulsations comprise a dominant component of the time-series signals. In one embodiment, a time-series signal is obtained by averaging all pixel values in the isolated region of exposed skin to obtain a channel average on a per-frame basis. A global channel average is computed, for each channel, by adding the channel averages across multiple image frames and dividing by the total number of frames comprising the batch. The channel average is then subtracted from the global channel average and the result is divided by a global channel standard deviation to obtain the time-series signal. The time-series signal may be filtered with a cutoff frequency defined as a function of a frequency of the subject's cardiac pulse. The time-series signal may be detrended to remove non-stationary components. Automatic peak detection may also be performed on the filtered signal. A VPG signal corresponding to the subject's cardiac function is extracted from the time-series signal.

“Extracting a continuous VPG signal” means to perform signal separation on the time-series signals to extract a continuous videoplethysmographic signal for the subject. Methods for extracting a plethysmographic signal from a time-series signal are disclosed in the above-incorporated references by Mestha and Mestha et al. Independent component analysis (ICA) can be used to recover VPG signals from a time-series signal. ICA is a decomposition technique for uncovering independent source signal components from a set of observations that are composed of linear mixtures of underlying sources, i.e., independent components of the observed data. Constrained source separation is an independent component analysis method for separating time-series signals into additive sub-components using a reference signal as a constraint. In one embodiment, the reference signal has a frequency range that approximates a frequency range of the subject's cardiac pulse. Not all constraints can be used for constrained independent component analysis (cICA) because some constraints infringe classical ICA equivariant properties. Constraints that define or restrict the properties of the independent components should not infringe the independence criteria. Additional conditions can be incorporated using, for example, sparse decomposition of signals or fourth-order cumulants into the contrast function, to help locate the global optimum separating the components. The obtained signal is converted to zero-mean unit variance. A filtering step is performed in order to improve peak detection accuracy. HR signals can be filtered using, for example, a moving average filter with a suitable moving window of size N frames. One example moving average filter is given as:

$$y ( n ) = \frac{1}{N} {\sum }_{1}^{N} x ( n - i )$$

where N is the number of frames in a moving window, x is the unfiltered plethysmographic signal, y is the filtered plethysmographic signal, n is the current frame i is the index designating the moving frame. Additional corrections may be necessary based on estimating the average of the amplitudes obtained from previous peaks. The VPG signal can also be filtered using, for example, an FFT-based phase preservation filter, a zero-phase digital filter, a linear time invariant (LTI) filter, a linear time varying (LTV) filter, a finite impulse response (FIR) filter, an infinite impulse response (IIR) filter, or a non-linear filter such as a median filter.

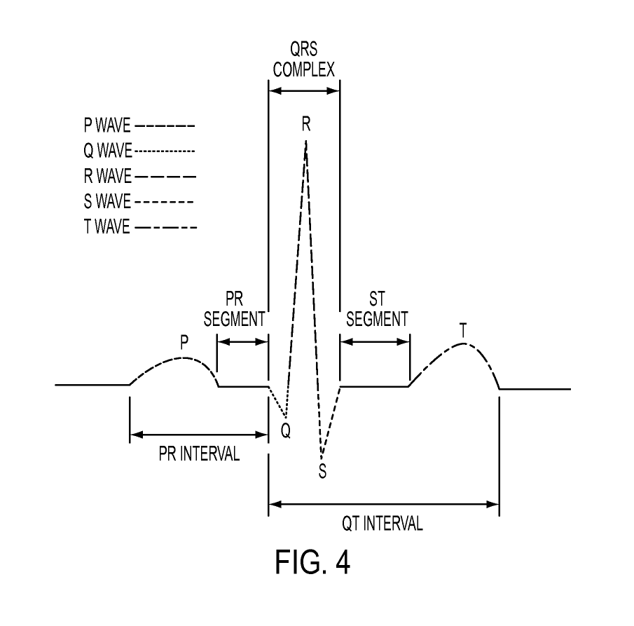

An “electrocardiogram (ECG) signal” (alternatively, EKG, from the Greek “kardia”, meaning heart) is a signal obtained from a sensor which senses the electrical activity of the heart. FIG. 1 shows an example ECG device 101 with a sensor 104 attached to the subject's chest. The sensor 104 may be attached to another part on the body where an ECG signal can be obtained. An example ECG signal 105 measured by the sensor 104 is shown being displayed on the display screen of the ECG device. The ECG signals may be communicated to a remote device via a wireless communication element 106, shown as an antenna. FIG. 4 shows a portion of an example ECG signal 400 for normal sinus rhythm. Although shown as a separate device, it should be appreciated that various functionality of the ECG device 101, the sensor 104, and the video imaging device 102 may be integrated into a single device. Such an integrated device may be further enabled to process image frames, time-series signals, as well as VPG and ECG signals, in accordance with the teachings hereof, to determine a PTT for the subject. Such a composite device may be, for example, a smartphone, an iPad, a tablet-PC, a laptop, or a computer workstation. ECG signals are well understood by cardiac function specialists. For a further discussion of ECG signals, the reader is directed to the introductory text: “EKGs for the Nurse Practitioner and Physician Assistant”, Maureen Knechtel, Springer Publishing Co. (2013), ISBN-13: 978-0826199560.

“Receiving an ECG signal” is intended to be widely construed and includes: retrieving, capturing, acquiring, or otherwise obtaining an ECG signal for processing. The ECG signal can be retrieved from a memory or storage device of the ECG device or retrieved from a media such as a CDROM or DVD. ECG signals can be obtained from a remote device over a network or downloaded from a web-based system or application which makes such signals available for processing. In accordance with the teachings hereof, the VPG and ECG signals are used to determine the transit time of an arterial pulse pressure wave.

“Pulse transit time (PTT)” is the time it takes an arterial pulse pressure wave to travel between two points. An arterial pulse pressure wave is generated when the left ventricle of the heart contracts and pushes a volume of blood out the ascending aorta into the systemic arteries. The repeated push of this blood volume generates a pulsating wave. The PTT is therefore the time taken for the arterial pulse pressure wave, which is originated from the left ventricle, to propagate through the arterial network to the region of exposed skin where the VPG signal was obtained. As disclosed herein, an instantaneous PTT is determined by computing a phase difference, dø, (in radians per second) between two reference points on the temporally synchronous ECG and VPG signals, as given by:

PTT=dø/f_{HR}

where f_{HR} is the frequency of the subject's cardiac pulse (in beats per minute). One method for estimating a cardiac pulse rate from a signal extracted from video is disclosed in the above-incorporated reference by Kyal et al.

In one embodiment, the reference point on the ECG signal is the peak point of the R wave and the reference point on the VPG signal is the peak of the peripheral pulse. It should be appreciated that the reference point on the VPG signal can be any characteristic point such as, for example, a maximum or a minimum point on the VPG signal, an average point between a maximum and a minimum on the VPG signal, a maximum of first derivative of the VPG signal derivative, and a maximum of a second derivative of the VPG signal. The PTT obtained is an instantaneous PTT and the PTT is obtained by averaging the instantaneous PTT over several cycles. PTT can be used to determine blood pressure, blood vessel dilation over time, blood vessel blockage, and blood flow velocity. Furthermore, PTT can be used as an indirect marker for assessing the occurrence of cardiac arrhythmia, cardiac stress, heart disease, and peripheral vascular disease. Since movement during video acquisition can adversely impact the extracted VPG signal, movement should be compensated for. In various embodiments hereof, a threshold level is set for movement.

A “threshold for movement” is a level of movement during video acquisition to determine whether motion artifacts may have been introduced into the video which will adversely impact the quality of the VPG signal extracted from the image frames during the time when the movement is determined to have occurred. A determination can be made whether a movement occurred during video acquisition by, for instance, using a motion detector or by visually observing the subject. Moreover, a determination can be made during batch processing of image frames by analyzing the isolated pixels. Such a determination can be made by: determining whether a location of a center pixel in the isolated region has changed relative to a fixed position; determining whether a size of the isolated region has changed relative to a size of a region isolated in at least one previous frame; determining whether a shape of the isolated region has changed relative to a shape of a region isolated in at least one previous frame; determining whether a color of pixels in the isolated region has changed relative to pixel colors of at least one previous frame; or by identifying a residual from frame differencing. If the movement is determined to be above the threshold level set for movement then movement has to be compensated in the current batch of image frames or these image frames need to be discarded. The threshold for movement may be pre-set by the user. The threshold may be based on a type of motion or a source of motion (i.e., by the subject or by the environment) or the time the movement occurred. The threshold may be automatically adjusted in real-time or manually adjusted by a user/technician as the video of the subject is being captured by the video imaging device. The exact level of this threshold for movement will largely depend on the application where the teachings hereof find their intended uses. Therefore, a discussion with respect to a particular threshold level is omitted. Other responses to movement exceeding the threshold include: initiating an alert signal that movement is excessive; signaling a medical professional that movement has occurred; adjusting a position of the video camera; adjusting a position of the subject; changing a frame rate of the video imaging device; swapping the video imaging device for another video camera; moving a position of the video imaging device; and stopping video acquisition altogether.

“Processing”, as used herein, broadly includes the application of any mathematical operation applied to data, according to any specific context, or for any specific purpose.

### Example Flow Diagram

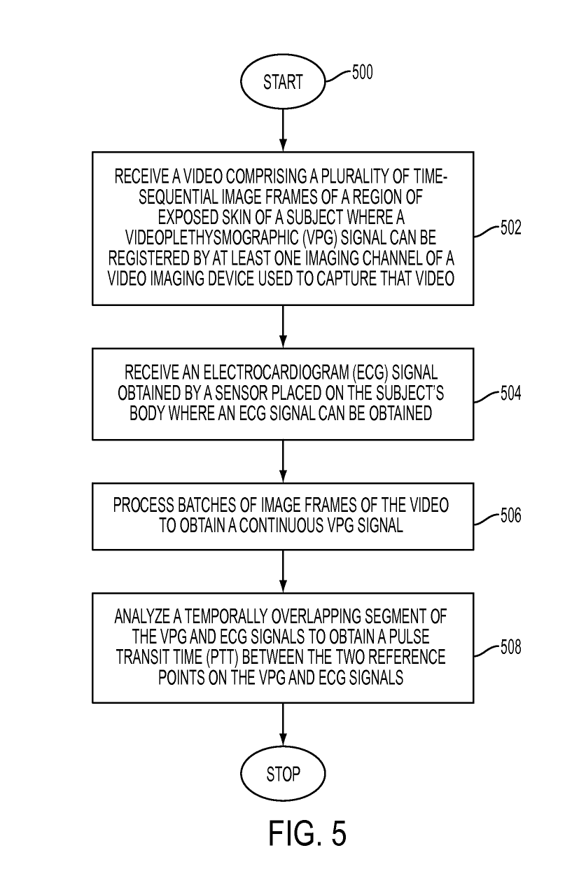

Reference is now being made to the flow diagram of FIG. 5 which illustrates one example embodiment of the present method for determining arterial pulse wave transit time for a subject. Flow processing begins at step 500 and immediately proceeds to step 502.

At step 502, receive a video comprising a plurality of time-sequential image frames of a region of exposed skin of a subject where a videoplethysmographic (VPG) signal can be registered by at least one imaging channel of the video imaging device used to capture that video.

At step 504, receive an electrocardiogram (ECG) signal obtained using a sensor placed on the subject's body where an ECG signal can be obtained.

At step 506, process batches of image frames of the video to obtain a continuous VPG signal for the subject.

At step 508, determine a pulse wave transit time between two reference points on the VPG and ECG signals. In this embodiment, further processing stops.

It should also be appreciated that the flow diagrams depicted herein are illustrative. One or more of the operations may be performed in a differing order. Other operations may be added, modified, enhanced, or consolidated. Variations thereof are intended to fall within the scope of the appended claims.

### Example Networked System

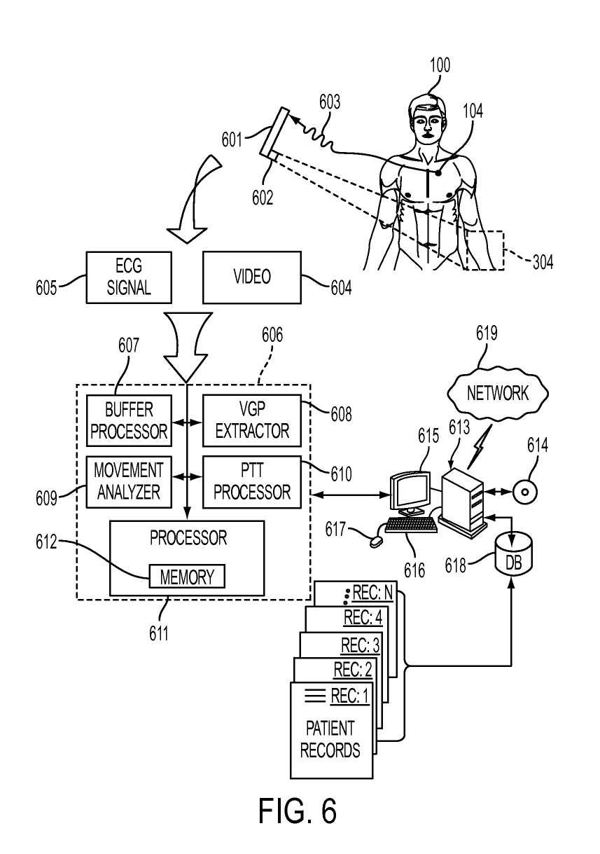

Reference is now being made to FIG. 6 which illustrates a block diagram of one example signal processing system 600 for performing various aspects of the teachings hereof.

In FIG. 6, a handheld wireless smartphone 601 utilizes the video camera 602 to acquire video of a region of exposed skin 304 of the subject 100. The region of exposed skin is shown in the field of view of the smartphone's video camera. Smartphone 601 is further configured with a sensor 104 shown attached to a chest area the subject to where an ECG signal can be obtained. The sensor is in communication with the smartphone via a wired connection (at 603). In other embodiments, the sensor is placed in wireless communication with the smartphone using a wireless protocol. In yet another embodiment, the smartphone itself is the ECG sensor and it obtains ECG signals by the smartphone being placed in contact with the skin surface. The video camera and the ECG sensor are configurable by a software application being executed by a processor in the smartphone. The application provides an icon widget in the form of a button which, when pressed by a user toughing the smartphone's touchscreen display, activates both the video camera and the ECG sensor to begin synchronous video capture and ECG signal acquisition. Turning the button OFF stops video capture and ECG signal acquisition. The image frames of the video 604 and the ECG signal 605 are communicated to the processing system 606.

In the embodiment of FIG. 6, the processing system 606 comprises a Batch Processor 607 which receives the image frames and processes batches of image frames to isolate pixels associated with the region of exposed skin (304). Batch Processor 607 further processes the isolated pixels to obtain a time-series signal for each batch. The time-series signal may be detrended and filtered as needed. The time-series signal for each batch is communicated to VPG Signal Extractor 608 wherein the time-series signal is processed to extract the VPG signal for the subject.

Movement Analyzer 609 continuously analyzes batches of image frames and makes a determination whether a movement occurred which exceeds a threshold level set of movement. A user may set the threshold level for movement on the smartphone prior to video acquisition and ECG signal capture, or may adjust the threshold dynamically during video and ECG signal acquisition by the smartphone. Adjustments can also be dynamically made by the user of the smartphone to the size of batches being processed and communicated to Batch Processor 607 so that a next batch of image frames can be processed with the dynamically adjusted batch size.

PTT Processor 610 receives the VPG signal and the ECG signal 605 and determines the transit time for the arterial pulse wave. Central Processor (CPU) 611 retrieves machine readable program instructions from Memory 612. The CPU and Memory, alone or in conjunction with other processors and memory, may be configured to assist or otherwise facilitate the functionality of any of the modules of system 606.

Processing system 606 is shown in communication with a workstation 613. A computer case of the workstation houses various components such as a motherboard with a processor and memory, a network card, a video card, a hard drive capable of reading/writing to machine readable media 614 such as a floppy disk, optical disk, CD-ROM, DVD, magnetic tape, and the like, and other software and hardware needed to perform the functionality of a computer workstation. The workstation further includes a display device 615, such as a CRT, LCD, or touchscreen device, for displaying information, video image frames, VPG signals, ECG signals, computed values, medical information, results, and the like. A user can view any of that information and make a selection from menu options displayed thereon or directly from the smartphone. Keyboard 616 and mouse 617 effectuate a user input or selection. The workstation implements a database in storage device 618 wherein patient records are stored, manipulated, and retrieved in response to a query. Such records, in various embodiments, take the form of patient medical history stored in association with information identifying the patient along with medical information. Although the database is shown as an external device, the database may be internal to the workstation mounted, for example, on a hard disk therein.

It should be appreciated that the workstation of FIG. 6 has an operating system and other specialized software configured to display alphanumeric values, menus, scroll bars, dials, slideable bars, pull-down options, selectable buttons, and the like, for entering, selecting, modifying, and accepting information needed for processing image frames, time-series signals, VPG and ECG signals in accordance with the teachings hereof. The workstation is further enabled to display the image frames comprising the video as well as the ECG signals captured by the sensor 104. In other embodiments, a user or technician uses the user interface of the workstation or the smartphone to identify one or more regions of exposed skin, set parameters, select image frames, view and analyze signals, and the like. These selections may be stored/retrieved in storage devices 614 and 618. Default settings and initial parameters can be retrieved from any of the storage devices shown.

Although shown as a desktop computer, it should be appreciated that the workstation can be a laptop, mainframe, or a special purpose computer such as an ASIC, circuit, or the like. The embodiment of the workstation of FIG. 6 is illustrative and may include other functionality known in the arts. Any of the components of the workstation may be placed in communication with the processing system 606 or any devices in communication therewith. Any of the modules and processing units of system 606 can be placed in communication with the database 618 and/or computer readable media 614 and may store/retrieve therefrom data, variables, records, parameters, functions, and/or machine readable/executable program instructions, as needed to perform their intended functions. Each of the modules of the video processing system 606 may be placed in communication with one or more remote devices over network 619.

It should be appreciated that some or all of the functionality performed by any of the modules or processing units of system 606 can be performed, in whole or in part, by the workstation placed in communication with the smartphone 601 over network 619. It should be understood that any of the functionality performed by the processing system 606 and/or the workstation 613 may be performed, in whole or in part, by the smartphone 601. The embodiment shown should not be viewed as limiting the scope of the appended claims strictly to that configuration. Various modules may designate one or more components which may, in turn, comprise software and/or hardware designed to perform the intended function.

### Performance Results

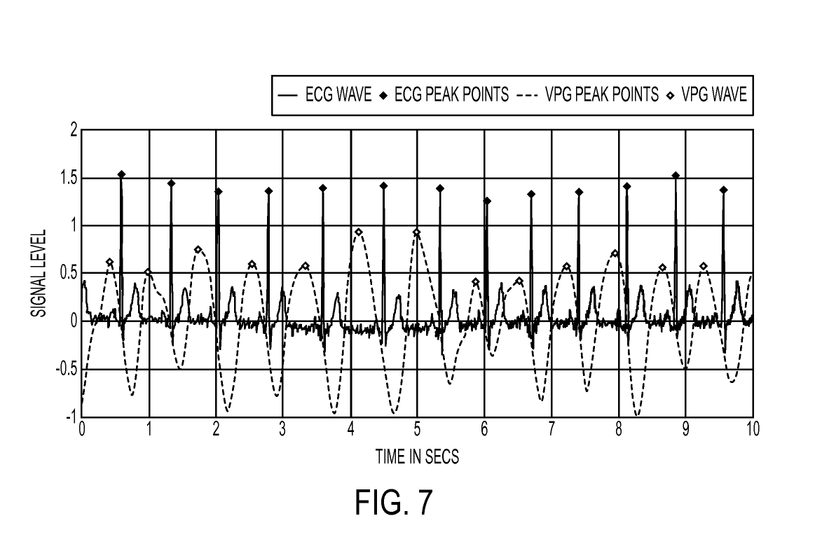

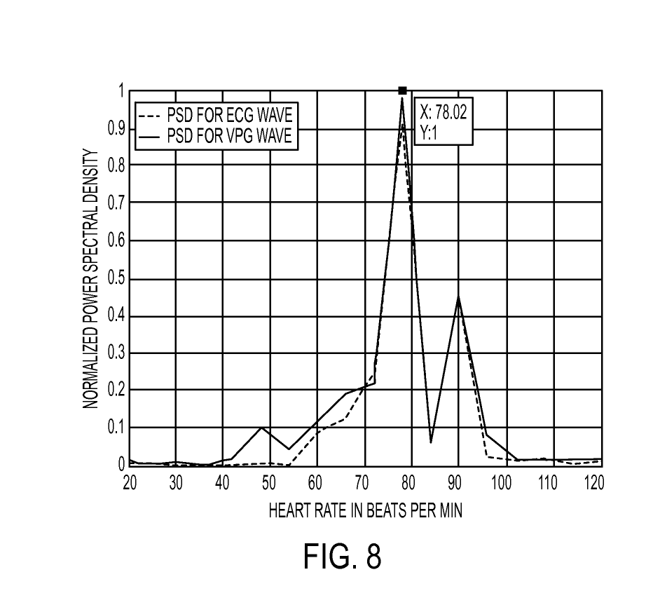

A single-lead ECG signal was recorded for a subject using a sensor with a sampling rate of about 500 Hz. At the same time, video was captured of a facial region of the subject using a video camera with a capability of recording 120 frames per second. The ECG sensor was distal to the facial region. Image frames of the captured video were processed and a VPG signal extracted. FIG. 7 shows a 10 second portion of a VPG signal overlaid on a simultaneously acquired ECG signal. PTT was determined by estimating an average time interval between peak-to-peak points between two waveforms over the duration of the segment. Other characteristic points between the ECG and VPG signals could have alternatively been used for PTT determination. For the signals shown in FIG. 7, we found the time interval to be about 248 ms. These signals were manually synchronized but automated synchronization could have been employed. Heart rate was found by estimating the power spectral density of the VPG signals. Alternatively, heart rate can be found by estimating a peak-to-peak difference of the VPG signal or obtained from analyzing the ECG signals. In this instance, the subject's heart rate was determined to be 1.3 Hz or about 78 bpm. FIG. 8 shows the normalized power spectral density for ECG and VPG signals v/s cardiac pulse frequency in beats per minute (bpm).

### Various Embodiments

It will be appreciated that the above-disclosed and other features and functions, or alternatives thereof, may be desirably combined into other different systems or applications. The teachings hereof can be implemented in hardware or software using any known or later developed systems, structures, devices, and/or software by those skilled in the applicable art without undue experimentation from the functional description provided herein with a general knowledge of the relevant arts.

One or more aspects of the methods described herein are intended to be incorporated in an article of manufacture which may be shipped, sold, leased, or otherwise provided separately either alone or as part of a product suite or a service. Presently unforeseen or unanticipated alternatives, modifications, variations, or improvements may become apparent and/or subsequently made by those skilled in this art which are also intended to be encompassed by the following claims.

The teachings of any publications referenced herein are each hereby incorporated by reference in their entirety.

What is claimed is:

1. A method for determining pulse wave transit time for a subject, comprising:
receiving a video acquired by a video imaging device, said video comprising a plurality of time-sequential image frames of a region of exposed skin of a subject where a videoplethysmographic (VPG) signal can be registered by at least one imaging channel of said video imaging device;
receiving an electrocardiogram (ECG) signal obtained using at least one sensor placed on said subject's body where an ECG signal can be obtained;
extracting a continuous VPG signal from batches of said image frames; and
processing temporally overlapping VPG and ECG signals to obtain a pulse transit time between a reference point on said VPG signal and a reference point on said ECG signal.

2. The method of claim 1, wherein said video imaging device is any of: a monochrome video camera, a color video camera, an infrared video camera, a multispectral video imaging device, a hyperspectral video imaging device, and a hybrid video imaging device comprising any combination hereof.

3. The method of claim 1, wherein, in advance of processing said image frames, further comprising compensating for any of: a motion induced blur, an imaging blur, and slow illuminant variation.

4. The method of claim 1, wherein processing said image frames to obtain said VPG signal comprises:
isolating pixels in said image frames associated with said region of exposed skin;
processing said isolated pixels to obtain a time-series signal; and
extracting, from said time-series signal, a VPG signal for said subject.

5. The method of claim 4, wherein pixels are isolated in said image frames using any of: pixel classification, object identification, thoracic region recognition, color, texture, spatial features, spectral information, pattern recognition, face detection, facial recognition, and a user input.

6. The method of claim 1, wherein, in advance of extracting said VPG signal from said time-series signal, further comprising any of:
detrending said time-series signal to remove non-stationary components;
filtering said time-series signal with a cutoff frequency defined as a function of a frequency of said subject's cardiac pulse; and
performing automatic peak detection on said filtered signal.

7. The method of claim 1, wherein, in advance of processing said VPG and ECG signals to obtain said pulse wave transit time, further comprising temporally synchronizing said VPG and ECG signal acquisition.

8. The method of claim 1, wherein said reference point on said ECG signal is a peak point of a R wave and said reference point on said VPG signal is a characteristic point on said VPG signal comprising any of: a maximum, a minimum, an average point between a maximum and a minimum, a maximum of a VPG signal derivative, and a maximum of a second derivative.

9. The method of claim 1, wherein both said video imaging device and said ECG sensor are integrated into any of: a smartphone, an iPad, a tablet-PC, a laptop, and a computer workstation.

10. The method of claim 1, further comprising determining whether a movement occurred during acquisition of said video image frames.

11. The method of claim 1, further comprising determining, from said pulse transit time, any of: blood pressure, blood vessel dilation over time, blood vessel blockage, and blood flow velocity.

12. The method of claim 11, further comprising determining an occurrence of any of: cardiac arrhythmia, cardiac stress, heart disease, and peripheral vascular disease.

13. The method of claim 1, further comprising communicating said pulse transit time to any of: a storage device, a display device, and a remote device over a network.

14. A system for determining arterial pulse wave transit time for a subject, the system comprising:
a processor in communication with a memory and storage device, said processor executing machine readable instructions for performing:
receiving a video acquired by a video imaging device, said video comprising a plurality of time-sequential image frames of a region of exposed skin of a subject where a videoplethysmographic (VPG) signal can be registered by at least one imaging channel of said video imaging device;
receiving an electrocardiogram (ECG) signal obtained using at least one sensor placed on said subject's body where an ECG signal can be obtained;
extracting a continuous VPG signal from batches of said image frames; and
processing temporally overlapping VPG and ECG signals to obtain a pulse transit time between a reference point on said VPG signal and a reference point on said ECG signal.

15. The system of claim 14, wherein said video imaging device is any of: a monochrome video camera, a color video camera, an infrared video camera, a multispectral video imaging device, a hyperspectral video imaging device, and a hybrid video imaging device comprising any combination hereof.

16. The system of claim 14, wherein, in advance of processing said image frames, further comprising compensating for any of: a motion induced blur, an imaging blur, and slow illuminant variation.

17. The system of claim 14, wherein processing said image frames to obtain said VPG signal comprises:
isolating pixels in said image frames associated with said region of exposed skin;
processing said isolated pixels to obtain a time-series signal; and
extracting, from said time-series signal, a VPG signal for said subject.

18. The system of claim 17, wherein pixels are isolated in said image frames using any of: pixel classification, object identification, thoracic region recognition, color, texture, spatial features, spectral information, pattern recognition, face detection, facial recognition, and a user input.

19. The system of claim 14, wherein, in advance of extracting said VPG signal from said time-series signal, further comprising any of:
detrending said time-series signal to remove non-stationary components;
filtering said time-series signal with a cutoff frequency defined as a function of a frequency of said subject's cardiac pulse; and
performing automatic peak detection on said filtered signal.

20. The system of claim 14, wherein, in advance of processing said VPG and ECG signals to obtain said pulse wave transit time, further comprising temporally synchronizing said VPG and ECG signal acquisition.

21. The system of claim 14, wherein said reference point on said ECG signal is a peak point of a R wave and said reference point on said VPG signal is a characteristic point on said VPG signal comprising any of: a maximum, a minimum, an average point between a maximum and a minimum, a maximum of a VPG signal derivative, and a maximum of a second derivative.

22. The system of claim 14, wherein both said video imaging device and said ECG sensor are integrated into any of: a smartphone, an iPad, a tablet-PC, a laptop, and a computer workstation.

23. The system of claim 14, further comprising determining, from said pulse transit time, any of: blood pressure, blood vessel dilation over time, blood vessel blockage, and blood flow velocity.

24. The system of claim 23, further comprising determining an occurrence of any of: cardiac arrhythmia, cardiac stress, heart disease, and peripheral vascular disease.

25. The system of claim 14, further comprising communicating said pulse transit time to any of: a storage device, a display device, and a remote device over a network.
